(19) United States
(12) Patent Application Publication
(10) Pub. No.: US 2021/0393191 A1
(43) Pub. Date: Dec. 23, 2021

(54) APPARATUSES INCLUDING FEATURES FOR PREDICTING PRESSURE WOUND FORMATION

(71) Applicant: TOYOTA MOTOR NORTH AMERICA, INC., Plano, TX (US)

(72) Inventor: Alexander Espiritu, Frisco, TX (US)

(73) Assignee: TOYOTA MOTOR NORTH AMERICA, INC., Plano, TX (US)

(21) Appl. No.: 16/908,268

(22) Filed: Jun. 22, 2020

(51) Int. Cl. A61B 5/00; A61G 5/10; A61B 5/01; G06K 9/62; H04N 5/225; H04N 7/18; H04N 5/232; G06K 9/46

(52) U.S. Cl. CPC A61B 5/447; A61G 5/1045; A61B 5/6894; A61B 5/015; A61B 5/0008; A61B 5/7275; A61B 2562/18; H04N 5/2253; H04N 7/18; H04N 5/232; G06K 9/4652; A61G 2203/46; A61B 2562/0276; G06K 9/6202

(57) ABSTRACT

Apparatuses including features for predicting pressure wound formation are disclosed. A wheelchair apparatus includes a seat having a surface that contacts a subject when the subject is sitting in the wheelchair apparatus. The surface includes a thermochromic layer having one or more materials that cause the thermochromic layer to change to a first color upon application of thermal energy radiating from the subject that corresponds to a baseline body temperature and change to a second color that is different from the first color upon application of thermal energy radiating from the subject that corresponds to a temperature that exceeds a threshold difference from the baseline body temperature. The second color is indicative of a potential pressure wound.

### TECHNICAL FIELD

The present disclosure generally relates to wheelchair apparatuses and, more specifically, to wheelchair apparatuses that include features that assist a user in determining areas containing potential pressure wounds on a subject based on skin temperature.

### BACKGROUND

Powered wheelchair apparatuses are known that are used to transport persons from one location to another. Transportation may be the primary operation of the powered wheelchair apparatuses. For some subjects, the powered wheelchair apparatuses may be an integral part of everyday life, and the subjects may sit in the powered wheelchairs for long periods of time. As a result, of this prolonged sitting in the powered wheelchair, pressure wounds can develop if the user is not regularly repositioned. However, existing wheelchair apparatuses are not able to continuously monitor a subject while the subject is seated and also are not able to provide an at-a-glance view of potential areas on the subject where pressure wounds may be developing.

### SUMMARY

In one aspect, a wheelchair apparatus includes a seat having a surface that contacts a subject when the subject is sitting in the wheelchair apparatus. The surface includes a thermochromic layer having one or more materials that cause the thermochromic layer to change to a first color upon application of thermal energy radiating from the subject that corresponds to a baseline body temperature and change to a second color that is different from the first color upon application of thermal energy radiating from the subject that corresponds to a temperature that exceeds a threshold difference from the baseline body temperature. The second color is indicative of a potential pressure wound.

In another aspect, a person support apparatus includes a body and a thermochromic layer disposed on a portion of the body that contacts a subject when the subject is supported by the person support apparatus. The thermochromic layer includes one or more materials that cause the thermochromic layer to change to a first color upon application of thermal energy radiating from the subject that corresponds to a baseline body temperature and change to a second color that is different from the first color upon application of thermal energy radiating from the subject that corresponds to a temperature that exceeds a threshold difference from the baseline body temperature. The second color is indicative of a potential pressure wound.

In yet another aspect, a wheelchair apparatus includes a seat having a surface that contacts a subject when the subject is sitting in the wheelchair apparatus, a plurality of temperature sensors disposed on the surface of the seat, and a wheelchair control system. The plurality of temperature sensors are arranged in a grid configuration such that each one of the plurality of temperature sensors senses a particular portion of the surface of the seat. The wheelchair control system includes a processor and a non-transitory, processor-readable memory module containing programming instructions thereon that, when executed, cause the processor to receive baseline temperature data from at least one of the plurality of temperature sensors, the baseline temperature data indicating a first temperature sensed when the subject sits in the wheelchair apparatus, receive subsequent temperature data from at least one of the plurality of temperature sensors, determine if the subsequent temperature data indicates a temperature that exceeds a threshold difference from the baseline temperature data, and when the subsequent temperature data indicates the temperature exceeding the threshold difference, determine a location of the at least one of the plurality of temperature sensors and transmit a notification pertaining to the exceeded threshold and the location.

These and additional objects and advantages provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts a perspective view of an illustrative wheelchair apparatus having one or more pressure wound indicator surfaces according to one or more embodiments shown and described herein;

FIG. 2 depicts a cutaway side view of an illustrative pressure wound indicator surface according to one or more embodiments shown and described herein;

FIG. 3 depicts a cutaway side view of another illustrative pressure wound indicator surface according to one or more embodiments shown and described herein;

FIG. 4 depicts an illustrative grid arrangement of temperature sensors on a surface according to one or more embodiments shown and described herein;

FIG. 5 schematically depicts a display for use with the wheelchair apparatus of FIG. 1 according to one or more embodiments shown and described herein; and

FIG. 6 diagrammatically illustrates a wheelchair control system for use with the wheelchair apparatus of FIG. 1 according to one or more embodiments shown and described herein.

### DETAILED DESCRIPTION

The systems and methods described herein generally relate to wheelchair apparatuses that include various features that assist users in determining the potential for pressure wound formation and address the pressure wound issues as soon as possible. The features of the systems and methods described herein that assist users generally function by providing an indicator regarding an elevated body temperature at a particular location of contact between the body of a subject and a surface of the wheelchair. The features may be passive, such as a surface having a thermochromic dye layer embedded therein, or may be active, such as a surface having a plurality of electronic temperature sensors. The various features can be integrated into the wheelchair apparatuses so as to not interfere with ordinary usage of the wheelchair apparatuses, while being integrated into a seat structure of the powered wheelchair apparatuses, such as into a seat, armrests, and the like.

A “wound” generally refers to tissue injury on a subject, which may include an opening in the subject's skin or may include unbroken skin that exhibits other injury characteristics, such as a contusion, redness, or the like. Pressure wounds, which may be one type of wound, may also be referred to as pressure sores, pressure ulcers, decubitus ulcers, and bedsores, are injuries to skin and underlying tissue resulting from prolonged pressure on the skin. Pressure wounds can develop on skin that covers bony areas of the body, such as, but not limited to, heels, ankles, hips, and tailbone. As such, subjects may develop pressure wounds on skin over their tailbone, buttocks, shoulder blades, spine, backs of arms and legs where resting against a portion of the wheelchair apparatus, and the like. Subjects at risk for pressure wounds sometimes have medical conditions that limit their ability to change positions or cause them to spend most of their time motionless in a bed or a chair (e.g., a wheelchair). Pressure wounds can develop over hours or days. One illustrative warning sign of pressure wounds includes, but is not limited to, an area of a subject's skin that feels cooler or warmer to the touch than other areas. Accordingly, the wheelchair apparatuses depicted and described herein can sense a localized change in temperature and/or provide an indicator of a localized change in temperature so that individuals can be notified and an action can be undertaken to avoid worsening conditions or to treat an affected area. While the wheelchair apparatuses described herein may generally be aimed at a target area that includes a pressure wound, it should be understood that other objects, such as surgical scars, lesions (e.g., moles), anatomical features surrounding the wound, and/or the like may also be imaged without departing from the scope of the present disclosure. In addition, the present disclosure references a “target object.” It should be understood that when various components of the wheelchair apparatuses described herein are aimed at a wound, the target object may be the wound. As such, the terms “target object” and “wound” may be used interchangeably herein. In addition, the term “target area” refers to an area as a whole, including the target object.

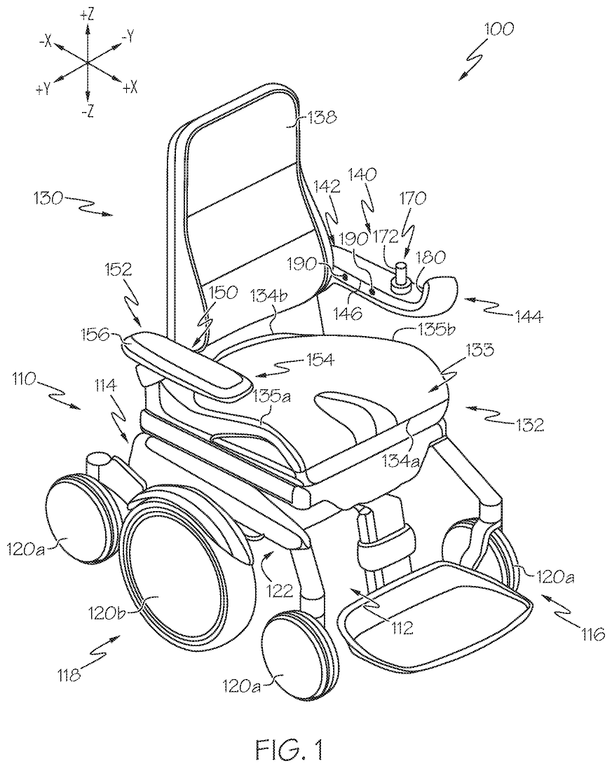

As used herein, the term “longitudinal direction” refers to the forward-rearward direction of the wheelchair apparatus (i.e., in the +/−X direction of the coordinate axes depicted in FIG. 1). The term “lateral direction” refers to the cross-direction (i.e., along the y-axis of the coordinate axes depicted in FIG. 1), and is transverse to the longitudinal direction. The term “vertical direction” refers to the upward-downward direction of the system (i.e., in the +/−Z direction of the coordinate axes depicted in FIG. 1). As used herein, “upper” or “top” is defined as generally being towards the positive Z direction of the coordinate axes shown in the drawings. “Lower” or “bottom” is defined as generally being towards the negative Z direction of the coordinate axes shown in the drawings.

Referring to FIG. 1, a wheelchair apparatus 100 generally includes a base component 110 and a chair component 130. The wheelchair apparatus 100 may be a powered wheelchair (as depicted in the embodiment of FIG. 1), a manual wheelchair, or a power assist wheelchair. Other types of wheelchairs are contemplated and included within the scope of the present disclosure. Further, while the present disclosure specifically relates to sensing in a wheelchair apparatus 100, the present disclosure is not limited to such. That is, any other person support apparatus, including, but not limited to, beds, stretchers, gurneys, chairs, benches, seats, and/or the like may incorporate the components described herein without departing from the scope of the present disclosure.

Still referring to FIG. 1, the base component 110 includes a wheelchair body 112 including a rearward support portion 114 and a forward support portion 116. The base component 110 may include a plurality of wheels 118. In the embodiment of FIG. 1 where the wheelchair apparatus 100 is a powered wheelchair, the plurality of wheels 118 may include one or more unpowered wheels 120a and/or one or more powered wheels 120b. A suspension 122 may be provided for the unpowered wheels 120a that can absorb energy when the unpowered wheels 120a are in use.

The chair component 130 of the wheelchair apparatus 100 generally includes a seat 132, a back 138, a first armrest 140, and a second armrest 150. In some embodiments, the first armrest 140 extends a length in a forward direction (e.g., in the +x direction of the coordinate axes of FIG. 1) such that a proximal end 142 of the first armrest 140 is adjacent to the back 138 and a distal end 144 is spaced a distance apart from the proximal end 142. In some embodiments, the second armrest 150 extends a length in a forward direction (e.g., in the +x direction of the coordinate axes of FIG. 1) such that a proximal end 152 of the second armrest 150 is adjacent to the back 138 and a distal end 154 is spaced a distance apart from the proximal end 152. In some embodiments, an armrest cushion 146, 156 may be disposed on each of the armrests 140, 150 and may provide an increased height of the respective armrest 140, 150 for increased arm height when resting thereon, may provide cushioning to absorb at least a portion of the pressure applied when a subject's arm is resting thereon, and may also include one or more temperature sensing and/or indicating features thereon or embedded therein, as described in greater detail herein.

The chair component 130 generally includes the seat 132 coupled to the back 138. The seat 132 may include an upper surface 133, and may have at least a front edge 134a, a first side edge 135a, a second side edge 135b, and a rear edge 134b. The seat 132 may generally be planar such that the upper surface 133 thereof extends between each of the front edge 134a, the first side edge 135a, the second side edge 135b, and the rear edge 134b (e.g., extending along the x-axis and the y-axis of the coordinate axes of FIG. 1). In some embodiments, the seat 132 may be constructed of a material that can be shaped, molded, contoured, or the like. For example, the seat 132, particularly the upper surface 133 thereof, may be contoured to a posterior of a subject. In another example, the upper surface 133 of the seat 132 may be padded with a padding material. In some embodiments, the seat 132 may be constructed of a material that absorbs liquid (e.g., a wicking material), a coated material, or a material that does not absorb liquids such that any liquids deposited on a surface of the seat 132 either pool on the surface thereof, drip off the surface, or pass through the material. In some embodiments, the seat 132 may support a removable and/or disposable cushion or the like.

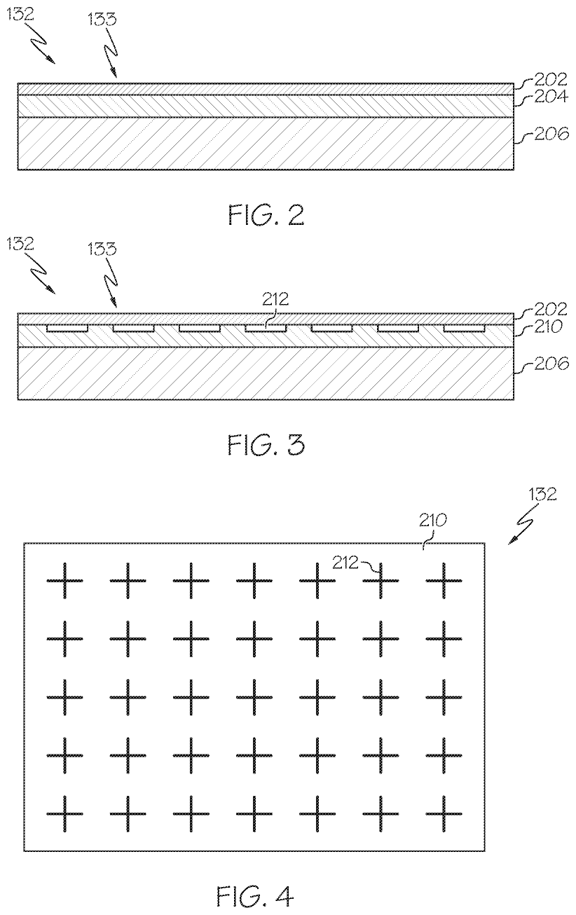

Turning to FIGS. 2-4, the seat 132 may have various features that allow for passive and/or active indicating of temperature variations, which can be used for the purposes of determining whether a subject sitting on the seat 132 is developing or has a pressure wound, as well as a target area containing the pressure wound. In the embodiment depicted in FIG. 2, the seat 132 may have a passive temperature sensing system. In the embodiment depicted in FIGS. 3 and 4, the seat 132 may have an active temperature sensing system. While the embodiments of FIG. 2 and FIGS. 3-4 are depicted as being separate (e.g., the embodiment of FIG. 2 only includes the passive temperature sensing system and FIGS. 3-4 only includes the active temperature sensing system), the present disclosure is not limited to such. That is, the seat 132 may include both a passive temperature sensing system and an active temperature sensing system in some embodiments.

Referring now to FIG. 2, the upper surface 133 of the seat 132 may have, for example, a thermochromic layer 204 disposed on or within a seat body 206 of the seat 132 in some embodiments such that the upper surface 133 of the seat 132 exhibits thermochromic properties. That is, the seat 132 includes the seat body 206 and the thermochromic layer 204, where the thermochromic layer 204 is the upper surface 133 of the seat 132. In some embodiments, a protective layer 202 may be disposed on the thermochromic layer 204 such that the upper surface 133 of the seat 132 is the protective layer 202. In such embodiments, the thermochromic layer 204 may be disposed between the components of the seat body 206 and the protective layer 202.

The seat body 206 generally includes various components that provide a supportive sitting surface. That is, the seat body 206 may include, but is not limited to, padding materials, springs or other biasing components, foam, structural support components, and/or the like. The various components of the seat body 206 (including components not specifically listed herein) that provide a supportive surface for a user to sit should generally be understood and are not described further herein.

The thermochromic layer 204 disposed on the seat body 206 is generally a layer including one or more materials, the layer coating the seat body 206 and exhibiting thermochromic properties. That is, the thermochromic layer 204 includes liquid crystals and/or leuco dyes that, when contacted with an object having a temperature that is different from an ambient temperature, change color at the point of contact. As such, when a subject sits on the seat 132, the subject's body temperature causes a localized change in color of the thermochromic layer 204 at each point of contact with the subject so that the thermochromic layer 204 changes to a first color at the point of contact with the seat 132. As will be described in greater detail hereinbelow, areas of the subject that are at risk for pressure wounds may exhibit a localized temperature that differs from the subject's body temperature (e.g., an elevated or reduced temperature), which causes the thermochromic layer 204 to change to a second color at the point of contact of the subject's body with the seat 132. As such, when the subject is removed from the seat 132, the thermochromic layer 204 can be visually inspected for areas that show the second color, thereby indicating areas on the user's body where there is a risk of pressure wound formation. It should be understood that such an indicator is possible even when the subject's body is covered with clothing, dressing gown, hospital garments, sheets, and/or the like, as the body heat radiating from the subject passes through the clothing, dressing gown, hospital garments, sheets, and/or the like to cause the color of the thermochromic layer 204 to change.

The thermochromic layer 204 may be particularly formulated from liquid crystals and/or leuco dyes such that a particular color change occurs at a first temperature range that corresponds to a subject's body temperature, and such that another particular color change occurs at a second temperature range that is greater than or less than the first temperature range (e.g., a particular temperature above or below a threshold difference from the first temperature range). The temperature ranges are not limited by this disclosure and can be particularly set by altering the chemical composition as necessary. In some embodiments, the temperature ranges may be selectable by a user. In some embodiments, the temperature ranges may be set according to recognized temperature ranges established by the medical community. That is, if medical journals, data, and/or the like indicate that certain temperature ranges are indicative of a typical human body temperature and other temperature ranges are indicative of potential pressure wound formation, the thermochromic layer 204 may be particularly formulated to change colors accordingly (e.g., to a first color at the first temperature range and to a second color at the second temperature range). For example, some liquid crystals are capable of displaying different colors at different temperatures. This change is dependent on selective reflection of particular wavelengths by a crystallic structure of the material as the material changes between a relatively low-temperature crystallic phase, through anisotropic chiral or twisted nematic phase, to a relatively high-temperature isotropic liquid phase. The nematic mesophase may have thermochromic properties, which restricts the effective temperature range of the material. The twisted nematic phase may have the molecules oriented in layers with regularly changing orientation, which gives them periodic spacing. The light passing through the crystal undergoes Bragg diffraction on these layers, and the wavelength with the greatest constructive interference is reflected back, which is perceived as a spectral color. A change in the crystal temperature can result in a change of spacing between the layers and therefore in the reflected wavelength. The color of the thermochromic liquid crystal can therefore continuously range from non-reflective (black) through the spectral colors to black again, depending on the temperature. In some embodiments, the relatively high temperature state will reflect a blue-violet color, while the relatively low-temperature state will reflect a red-orange color. Since blue is a shorter wavelength than red, this indicates that the distance of layer spacing is reduced by heating through the liquid-crystal state. Illustrative thermochromatic liquid crystals that may be used for the thermochromic layer 204 include, but are not limited to, cholesteryl nonanoate and a cyanobiphenyl. In embodiments, liquid crystals may be microencapsulated in the form of a suspension or the like.

In embodiments where leuco dyes are used, the leuco dyes may be mixed with various other compounds to display a color change (usually between the colorless leuco form and the colored form) in dependence on temperature. The dyes may be deposited on the seat body 206 in the form of microcapsules having a mixture sealed therein. An illustrative example of a leuco dye includes, but is not limited to, microcapsules having crystal violet lactone, weak acid, and a dissociable salt dissolved in dodecanol that is applied to the seat body 206. When the solvent is solid, the dye exists in its lactone leuco form. When the solvent melts due to the application of thermal energy (e.g., as a result of contact between the subject and the seat 132), the salt dissociates, the pH inside the microcapsule lowers, the dye becomes protonated, its lactone ring opens, and its absorption spectrum shifts, causing a color change. Illustrative examples of leuco dyes include, but are not limited to, spirolactones, fluorans, spiropyrans, and fulgides. Illustrative examples of acids include, but are not limited to, bisphenol A, parabens, 1,2,3-triazole derivates, and 4-hydroxycoumarin, and act as proton donors, changing the dye molecule between its leuco form and its protonated colored form. In some embodiments, the leuco dye may be used in combination with some other pigment, producing a color change between the color of the base pigment and the color of the pigment combined with the color of the non-leuco form of the leuco dye. In some embodiments, the leuco dye may be provided in the form of a thermochromic paint that is deposited on the seat body 206. Other materials and compositions that can be configured to change color when thermal energy is applied are contemplated and included in the scope of the present disclosure.

The thermochromic layer 204 provides a means of passively detecting potential areas on a subject that may be developing pressure wounds. The thermochromic layer 204 is passive because such detection of the potential areas is only possible when the subject gets off the seat 132 so that the upper surface 133 thereof is visible. That is, the thermochromic layer 204 changes to a first color in particular areas where there is contact between the subject and the seat 132 and also changes to a second color that is different than the first color in particular areas where the heat radiated from the subject is different from the user's baseline body temperature. Upon removal of the subject from the seat 132, the areas of the thermochromic layer 204 exhibiting the second color are viewable by a user (e.g., the subject, a caretaker, a family member, or the like) or imaged by an imaging device (as described herein), correlated to an area on the subject (e.g., the location of an area of interest on the subject is estimated based on the location on the thermochromic layer 204), and various measures can be taken (e.g., inspect the subject for pressure wounds, treat the subject, provide a notification, and/or the like).

The protective layer 202 is generally a layer of material that is deposited over the thermochromic layer 204 that protects the thermochromic layer 204 and/or the seat body 206 from damage. The material used for the protective layer 202 is generally not limited by the present disclosure and may generally be any thermally conductive material that is transparent such that the thermal energy radiating from the subject can pass therethrough to the thermochromic layer 204 to cause color change and such that the change in color can be visually detected therethrough. Illustrative materials may include, but are not limited to, ceramics such as aluminum oxide (Al₂O₃), boron nitride (BN), magnesium oxide (MgO), and/or the like, films that include polymers (e.g., polyethylene), graphene, and/or the like. Various materials that include such properties are contemplated and included within the scope of the present disclosure.

It should be understood that the protective layer 202 may be omitted in some embodiments such that only the thermochromic layer 204 is disposed on the seat body 206. In still other embodiments, the thermochromic layer 204 may be deposited within a portion of the seat body 206 (e.g., the seat body 206 may be impregnated with or partially constructed of the materials that provide thermochromic properties). Other combinations of the seat body 206 and the thermochromic materials of the thermochromic layer 204 that provide an ability to locally change in color depending on the temperature of various parts of the subject sitting on the seat 132 are contemplated and included in the scope of the present disclosure.

Referring to FIGS. 3 and 4, the upper surface 133 of the seat 132 may include a sensing layer 210 that is disposed on or within the seat body 206 of the seat 132 in some embodiments. That is, the seat 132 includes the seat body 206 and the sensing layer 210, where the sensing layer 210 is the upper surface 133 of the seat 132 or disposed on the upper surface 133 of the seat 132. As described in greater detail below, the sensing layer 210 includes a plurality of temperature sensors 212 that are arranged to actively detect temperature and transmit data corresponding to the detected temperature. As depicted in FIGS. 3-4, the temperature sensors 212 are arranged in a grid like fashion (e.g., a grid configuration) such that each sensor of the plurality of temperature sensors 212 senses a temperature of a particular area of the upper surface 133 of the seat 132. However, it should be understood that such an embodiment is merely illustrative and other, non-grid like configurations of the temperature sensors 212 are contemplated and included in the scope of the present disclosure.

The temperature sensors 212 may generally be any electronic device that is configured to determine a temperature at a particular location of the upper surface 133 of the seat 132, particularly when the seat 132 is occupied by a subject. Each one of the temperature sensors 212 may be a non-contact sensor such as an infrared (IR) sensor or a contact sensor such as a thermocouple or a thermistor. Other types of temperature sensing devices are also contemplated and included within the scope of the present disclosure.

The plurality of temperature sensors 212 as depicted in the embodiment of FIGS. 3 and 4 provide a means of actively detecting potential areas on a subject that may be developing pressure wounds. The plurality of temperature sensors 212 are active because such detection of the potential areas can occur while the subject is seated and can be continuously monitored as long as the subject is seated on the seat 132. Signals can be continuously transmitted to a processing device (as described in greater detail herein) for the purposes of determining a baseline body temperature of a subject and for determining an anomaly (e.g., a temperature that deviates from the baseline body temperature), determining the location of the anomaly based on the one or more sensors from the plurality of temperature sensors 212 that provided the temperature reading, and determining one or more actions to be taken as a result (e.g., transmitting a notification of the anomaly, transmitting a location of the anomaly, and/or the like).

The protective layer 202 may be the same protective layer of the embodiment of FIG. 2 or may be a different protective layer. That is, the protective layer 202 is generally a layer of material that is deposited over the sensing layer 210 including the plurality of temperature sensors 212 that protects plurality of temperature sensors 212 and/or the seat body 206 from damage. As previously described herein, the material used for the protective layer 202 is generally not limited by the present disclosure and may generally be any thermally conductive material. It should be understood that the protective layer 202 may be omitted in some embodiments such that only sensing layer 210 is disposed on the seat body 206. In still other embodiments, the sensing layer 210 may be disposed within a portion of the seat body 206 (e.g., the seat body 206 may be integrated with or partially constructed with the plurality of temperature sensors 212 therein). Other combinations of the seat body 206 and the sensing layer 210 that provide an ability to locally detect change in temperature from a baseline are contemplated and included in the scope of the present disclosure.

While the embodiments of FIGS. 2-4 generally relate to active and/or passive temperature sensing in the seat 132 of the wheelchair apparatus 100 (FIG. 1), the present disclosure is not limited to such. That is, referring again to FIG. 1 in addition to FIGS. 2-4, various other portions of the wheelchair apparatus 100 may be similarly configured to actively and/or passively sense a body temperature of a subject at any point of contact between the subject and the wheelchair apparatus 100 when the subject is sitting thereon, including, but not limited to, the back 138, the first armrest 140, the second armrest 150, leg supports (not depicted), and/or the like. For example, the back 138, the first armrest 140, the second armrest 150, leg supports (not depicted), and/or the like may also include a thermochromic layer 204 (e.g., a second thermochromic layer, a third thermochromic layer, etc.) and/or a sensing layer 210 (e.g., a second sensing layer, a third sensing layer, etc.).

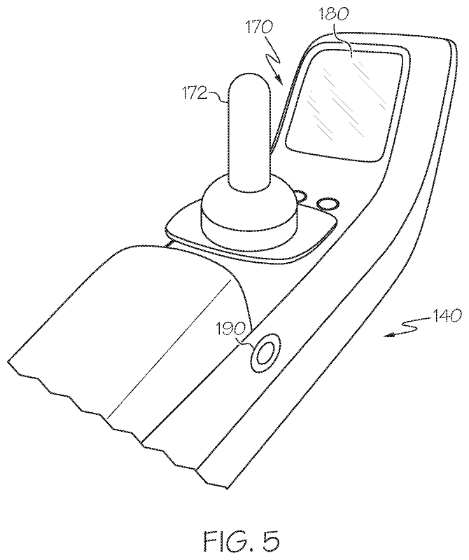

Referring to FIGS. 1 and 5, the wheelchair apparatus 100 further includes a number of usage features including, but not limited to, a user input device 170 that can be used to control various features of the wheelchair apparatus 100. In some embodiments, the user input device 170 may include a joystick 172 that can be manually moved about a plurality of axes in order to control various features of the wheelchair apparatus 100 (e.g., drive the wheelchair apparatus 100). In some embodiments, the joystick 172 may be located at a distal end 144 of the first armrest 140 and/or the distal end 154 of the second armrest 150 for ease in accessibility. While only depicted on the first armrest 140 in the embodiment of FIGS. 1 and 5, the joystick 172 may be provided on one or both of the armrests 140, 150. In some embodiments, the joystick 172 may be used to control a drive unit of the wheelchair apparatus 100.

In some embodiments, the wheelchair apparatus 100 may also include one or more imaging devices 190 that are positioned to capture an image of the upper surface 133 of the seat 132. As depicted in FIGS. 1 and 5, the imaging devices 190 are located in the first armrest 140 and the second armrest 150 (obscured from view in FIG. 1). However, other locations are contemplated, including locations on the wheelchair apparatus 100 and off the wheelchair apparatus 100 (e.g., imaging devices 190 disposed within a room or space in which the wheelchair apparatus 100 is located). As described in greater detail herein, the imaging devices 190 may generally be any electronic device that can capture an image (e.g., a visible spectrum image, an infrared image, and/or the like) of various portions of the wheelchair apparatus 100. The image data that results from the images captured by the imaging devices 190 can be usable to electronically determine whether the passive temperature sensing (e.g., the thermochromic layer 204 depicted in FIG. 2) indicates a change in temperature that might be indicative of pressure wounds, as well as a location thereof. Various hardware that provides such imaging capabilities should generally be understood and is not described in greater detail herein.

In some embodiments, a display 180 may be provided on the wheelchair apparatus 100. While only depicted on the first armrest 140 in the embodiment of FIGS. 1 and 5, the display 180 may be provided on one or both of the armrests 140, 150. As described below, the display 180 may be a touch screen and allow for user input in some embodiments. As such, the user input device 170 may include the display 180 in some embodiments.

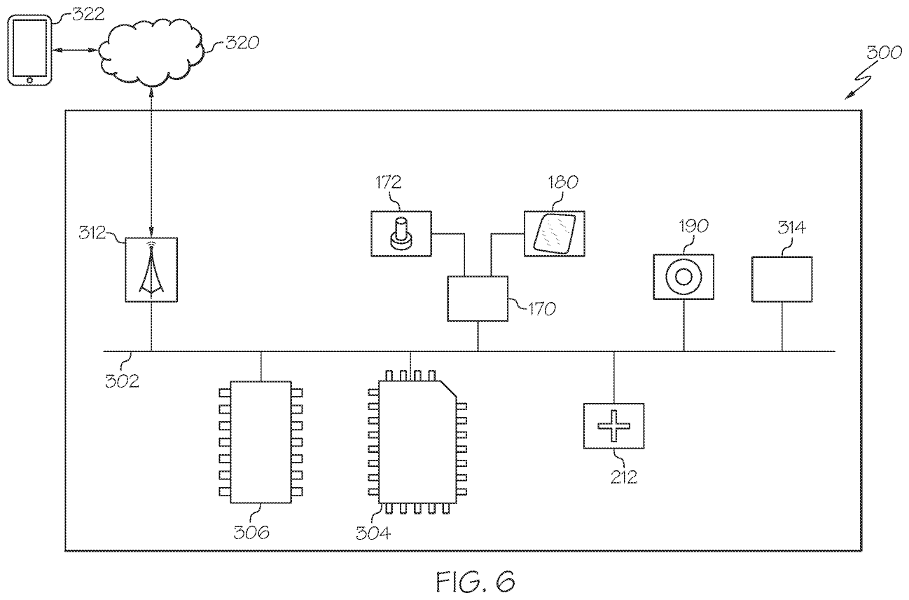

Referring now to FIG. 6, a wheelchair control system 300 of the wheelchair apparatus 100 (FIG. 1) is illustrated schematically. The wheelchair control system 300 includes a communication path 302, a processor 304, a memory module 306, and the user input device 170. The wheelchair control system 300 may further include the imaging devices 190, the plurality of temperature sensors 212, network interface hardware 312, and one or more drive components 314. As previously described herein, the user input device 170 may include the joystick 172 and/or the display 180.

The wheelchair control system 300 includes the communication path 302 that provides data interconnectivity between various modules disposed within the wheelchair control system 300. Specifically, each of the modules can operate as a node that may send and/or receive data. In some embodiments, the communication path 302 includes a conductive material that permits the transmission of electrical data signals to processors, memories, sensors, and actuators throughout the wheelchair control system 300. In some embodiments, the communication path 302 may be wireless and/or an optical waveguide. Components that are communicatively coupled may include components capable of exchanging data signals with one another such as, for example, electrical signals via conductive medium, electromagnetic signals via air, optical signals via optical waveguides, and the like.

The wheelchair control system 300 includes the processor 304 communicatively coupled with the memory module 306 over the communication path 302. The processor 304 may include any device capable of executing machine-readable instructions stored on a non-transitory computer-readable medium. The processor 304 may include one or more processors. Accordingly, each processor 304 may include a controller, an integrated circuit, a microchip, a computer, and/or any other computing device.

The memory module 306 is communicatively coupled to the processor 304 over the communication path 302. The memory module 306 may be configured as volatile and/or nonvolatile memory and, as such, may include random access memory (including SRAM, DRAM, and/or other types of RAM), flash memory, secure digital (SD) memory, registers, compact discs (CD), digital versatile discs (DVD), and/or other types of non-transitory computer-readable mediums. Depending on the particular embodiment, these non-transitory computer-readable mediums may reside within the wheelchair control system 300 and/or external to the wheelchair control system 300. The memory module 306 may be configured to store one or more pieces of logic, as described in more detail below. The memory module 306 may include one or more memory modules. The embodiments described herein may utilize a distributed computing arrangement to perform any portion of the logic described herein.

Embodiments of the present disclosure include logic stored on the memory module 306 that includes machine-readable instructions and/or an algorithm written in any programming language of any generation (e.g., 1GL, 2GL, 3GL, 4GL, and/or 5GL) such as, machine language that may be directly executed by the processor 304, assembly language, object-oriented programming (OOP), scripting languages, microcode, etc., that may be compiled or assembled into machine readable instructions and stored on a machine readable medium. Similarly, the logic and/or algorithm may be written in a hardware description language (HDL), such as logic implemented via either a field-programmable gate array (FPGA) configuration or an application-specific integrated circuit (ASIC), and their equivalents. Accordingly, the logic may be implemented in any conventional computer programming language, as pre-programmed hardware elements, and/or as a combination of hardware and software components. The logic may be configured such that, when completed by the processor 304, causes the processor 304 to complete the various processes described herein (e.g., processes relating to receiving data from the temperature sensors 212 and/or the imaging devices 190), determining a threshold, determining whether a temperature is elevated, determining a location of an elevated temperature, and completing one or more additional tasks (e.g., transmitting a notification).

The wheelchair control system 300 includes the user input device 170 coupled to the communication path 302 such that the communication path 302 communicatively couples the user input device 170 to other modules of the wheelchair control system 300. As described above, the user input device 170 may be controlled manually. In some embodiments, there may be multiple user input devices. The user input device 170 may be any device capable of transforming mechanical, optical, or electrical signals into a data signal capable of being transmitted with the communication path 302. Specifically, the user input device 170 may include any number of movable objects that transform physical motion into a data signal that can be transmitted over the communication path 302 such as, for example, a button, a switch, a knob, the joystick 172, a microphone, and/or the like. In some embodiments, the display 180 and the user input device 170 are combined as a single module and operate as a touchscreen user input device. However, it is noted that the display 180 and the user input device 170 may be separate from one another. In some embodiments, there may not be a display 180. The user input device 170 may allow a user (e.g., a subject) to control operation of the wheelchair apparatus 100 (FIG. 1) and/or view images, text, data and/or the like relating to the temperature sensing.

In some embodiments, the wheelchair control system 300 further includes network interface hardware 312 for communicatively coupling the wheelchair control system 300 with a network 320. The network interface hardware 312 can be communicatively coupled to the communication path 302 and can be any device capable of transmitting and/or receiving data via the network 320. Accordingly, the network interface hardware 312 can include a communication transceiver for sending and/or receiving any wired or wireless communication. For example, the network interface hardware 312 may include an antenna, a modem, LAN port, Wi-Fi card, WiMax card, mobile communications hardware, near-field communication hardware, satellite communication hardware and/or any wired or wireless hardware for communicating with other networks and/or devices. In one embodiment, the network interface hardware 312 includes hardware configured to operate in accordance with the Bluetooth wireless communication protocol. In another embodiment, network interface hardware 312 may include a Bluetooth send/receive module for sending and receiving Bluetooth communications to/from an external computing device 322 (e.g., data pertaining to temperature sensing as described herein).

The wheelchair control system 300 may communicate, through the network interface hardware 312, with the network 320 to communicatively couple the wheelchair control system 300 with the external computing device 322. In one embodiment, the network 320 is a personal area network that utilizes Bluetooth technology to communicatively couple the wheelchair control system 300 and the external computing device 322. In other embodiments, the network 320 may include one or more computer networks (e.g., a personal area network, a local area network, or a wide area network), cellular networks, satellite networks and/or a global positioning system and combinations thereof. Accordingly, the wheelchair control system 300 can be communicatively coupled to the network 320 via wires, via a wide area network, via a local area network, via a personal area network, via a cellular network, via a satellite network, etc. Suitable local area networks may include wired Ethernet and/or wireless technologies such as, for example, wireless fidelity (Wi-Fi). Suitable personal area networks may include wireless technologies such as, for example, IrDA, Bluetooth, Wireless USB, Z-Wave, ZigBee, and/or other near field communication protocols. Suitable personal area networks may similarly include wired computer buses such as, for example, USB and FireWire. Suitable cellular networks include, but are not limited to, technologies such as LTE, WiMAX, UMTS, CDMA, and GSM.

In some embodiments, the external computing device 322 may be included as a user input device. The external computing device 322 may include a processor 304 and a memory module. The processor 304 can execute logic to communicate with the wheelchair control system 300 in order to facilitate sending instructions to the wheelchair control system 300 from the external computing device 322 to control the wheelchair apparatus 100 (FIG. 1), as well as to receive data from the wheelchair apparatus 100 (e.g., data pertaining to temperature sensing). The external computing device 322 may be configured with wired and/or wireless communication functionality for communicating with the wheelchair control system 300. In embodiments described herein, the external computing device 322 may include mobile phones, smartphones, personal digital assistants, dedicated mobile media players, mobile personal computers, laptop computers, and/or any other mobile devices capable of being communicatively coupled with the wheelchair control system 300. It is noted, that in this embodiment, the wheelchair control system 300 may communicate with the external computing device 322 even while the external computing device 322 is remote from the wheelchair apparatus 100 (FIG. 1). In this way, the wheelchair apparatus 100 (FIG. 1) may be controlled with the external computing device 322 remotely from outside of the wheelchair apparatus 100 (FIG. 1).

The wheelchair control system 300 may further include the display 180 for providing visual output such as, for example, maps, navigation, entertainment, information, image data from the imaging devices 190, the plurality of temperature sensors 212, or a combination thereof. The display 180 is coupled to the communication path 302. Accordingly, the communication path 302 communicatively couples the display 180 to other modules of the wheelchair control system 300. The display 180 may include any medium capable of transmitting an optical output such as, for example, a cathode ray tube, light emitting diodes, a liquid crystal display, a plasma display, or the like. Moreover, the display 180 may be a touchscreen that, in addition to providing optical information, detects the presence and location of a tactile input upon a surface of or adjacent to the display 180. Accordingly, the display 180 may receive mechanical inputs directly upon the optical output provided by the display 180. As such, the display 180 may be included as a user input device. Additionally, it is noted that the display 180 can include a processor 304 and a memory module.

The wheelchair control system 300 may further include the imaging devices 190 coupled to the communication path 302 such that the communication path 302 communicatively couples the imaging devices 190 to other modules of the wheelchair control system 300. The imaging devices 190 may include any devices having an array of sensing devices (e.g., pixels) capable of capturing image data from an environment of the wheelchair apparatus 100 (FIG. 1). The imaging devices 190 may have any resolution. As noted above, image data received from the imaging devices 190 may be displayed on the display 180. In some embodiments, the wheelchair control system 300 may actuate the imaging devices 190 upon receiving instructions from the user input device 170.

The wheelchair control system 300 may further include the plurality of temperature sensors 212 coupled to the communication path 302 such that the communication path 302 communicatively couples the plurality of temperature sensors 212 to other modules of the wheelchair control system 300. As noted herein, information pertaining to the data received from the plurality of temperature sensors 212 may be displayed on the display 180 to indicate any areas exhibiting a temperature that is different from a baseline, so as to notify any users (e.g., the subject) of potential pressure wound formation. In some embodiments, the wheelchair control system 300 may actuate the plurality of temperature sensors 212 upon receiving instructions from the user input device 170.

The wheelchair control system 300 may further include one or more drive components 314 communicatively coupled over the communication path 302 to other wheelchair modules. The one or more drive components 314 may be configured to execute one or more driving modes of the wheelchair apparatus 100 (FIG. 1). For example, the one or more drive components 314 may provide power to one or more motors driving the wheels of the wheelchair apparatus 100 in response to one or more inputs received (e.g., inputs received via the user input device 170).

Referring to FIGS. 1-2 and 5-6, in operation, when the passive sensing according to the embodiment of FIG. 2 is utilized in addition to the imaging devices 190, the imaging devices 190 may capture a baseline image prior to a subject sitting in the wheelchair apparatus 100, the baseline image indicating a baseline state of the seat 132 when the thermochromic layer 204 is not exhibiting any color change, the baseline image being stored in the memory module 306 for later access. Images may be periodically obtained while the subject is sitting on the seat 132 to verify that the subject is sitting. When the subject gets off the seat 132, one or more images of the seat 132 are captured and used for comparison with the baseline image by the processor 304. In some embodiments, detection of the subject getting off the seat 132 may be completed by analyzing each periodically-obtained image (via image analysis software executed by the processor 304) to determine whether the subject is in the seat 132. In other embodiments, detection of the subject getting off the seat 132 may be as a result of a signal that is received by the processor 304 (e.g., the subject or another user presses a button on the user input device 170 indicating that the subject is getting off the seat 132, a pressure sensor located in the seat body 206 transmits a signal, and/or the like). Comparison of the captured image at the time the subject gets off the seat 132 with the baseline image may be completed by the processor 304 using image comparison software to determine whether any elevated temperature indicators are present. That is, the processor 304 may determine one or more areas exhibiting a first color change (indicating where the subject was sitting on the seat 132) and a second color change (indicating areas on the subject where a different temperature is observed). If the analysis of the images indicates no second color, no action may be taken or a message may be transmitted (e.g., to the external computing device 322), the message indicating that no areas of concern have been detected. If the analysis of the images indicates a second color in one or more areas, a determination of the location of the one or more areas may be made and a message may be transmitted (e.g., to the external computing device 322) that one or more areas of concern have been detected, as well as a location of the one or more areas of concern.

Referring to FIGS. 1 and 3-6, in operation, when the active sensing according to the embodiment of FIGS. 3-4 is utilized, the plurality of temperature sensors 212 may capture baseline temperature data when the subject sits in the wheelchair apparatus 100, the baseline temperature data indicating a baseline temperature of a subject at the areas where contact between the subject and the seat 132 are detected. That is, at least one of the plurality of temperature sensors 212 transmits temperature data upon receiving an indication to begin transmitting (e.g., if a pressure sensor senses the subject in the seat, the sensed temperature rises significantly from an ambient temperature, and/or a user actuates a button or the like indicating that the subject is sitting in the seat 132, a signal may be transmitted). In some embodiments, the baseline temperature data may be stored in the memory module 306 for later access. Temperature data may be periodically captured while the subject is sitting on the seat 132 for comparison with the baseline data by the processor 304. Comparison of the captured data with the baseline data may be completed by the processor 304 using comparison software to determine one or more areas exhibiting a variation in temperature that is greater than a threshold. The threshold is not limited by the present disclosure and may be any number of degrees above or below the baseline reading (including fractions of a degree). In some embodiments, the threshold may be selectable by a user. In some embodiments, the threshold may vary based on the baseline temperature. In some embodiments, the threshold may be set according to a recognized threshold established by the medical community. That is, if medical journals, data, and/or the like indicate that a threshold change of a certain amount (e.g., 1° C.) is indicative of potential pressure wound formation, the threshold may be set (e.g., via the user input device 170) to correspond to that amount. The location is determined based on a known location of the sensor(s) that reported the data as being greater than the threshold. If the analysis of the data indicates no change in temperature, no action may be taken or a message may be transmitted (e.g., to the external computing device 322), the message indicating that no areas of concern have been detected. If the analysis of the data indicates a change in temperature in one or more areas, a message may be transmitted (e.g., to the external computing device 322) that one or more areas of concern have been detected, as well as a location of the one or more areas of concern.

It should now be understood that the wheelchair apparatuses described herein include various features that assist users in determining the potential for pressure wound formation so that the users can address the pressure wound issues as soon as possible. The wheelchair apparatuses include features that actively and/or passively sense temperature deviations radiating from a subject's body when the subject is sitting on the seat of the wheelchair apparatuses and provide an indicator regarding an elevated body temperature at a particular location of contact between the body of a subject and a surface of the wheelchair. The passive features include a thermochromic layer and the active, such as a surface having a plurality of electronic temperature sensors. The various features can be integrated into the wheelchair apparatuses so as to not interfere with ordinary usage of the wheelchair apparatuses, while being integrated into a seat structure of the powered wheelchair apparatuses, such as into a seat, armrests, and the like.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. A wheelchair apparatus, comprising:
a seat comprising a surface that contacts a subject when the subject is sitting in the wheelchair apparatus, the surface comprising:
a thermochromic layer comprising one or more materials that cause the thermochromic layer to:
change to a first color upon application of thermal energy radiating from the subject that corresponds to a baseline body temperature, and
change to a second color that is different from the first color upon application of thermal energy radiating from the subject that corresponds to a temperature that exceeds a threshold difference from the baseline body temperature, the second color indicative of a potential pressure wound.

2. The wheelchair apparatus of claim 1, further comprising one or more imaging devices arranged to capture one or more images of the thermochromic layer.

3. The wheelchair apparatus of claim 2, further comprising:
a processor communicatively coupled to the one or more imaging devices; and
a non-transitory, processor-readable memory module containing programming instructions thereon that, when executed, cause the processor to:
direct the one or more imaging devices to capture a baseline image of the thermochromic layer,
store the baseline image,
receive an indication that the subject is no longer sitting in the wheelchair apparatus,
direct the one or more imaging devices to capture an additional image of the thermochromic layer, and
compare the baseline image with the additional image to determine whether any elevated temperature indicators are present.

4. The wheelchair apparatus of claim 3, wherein the programming instructions that, when executed, cause the processor to determine whether any elevated temperature indicators are present further cause the processor to:
determine the first color of the thermochromic layer,
determine that the second color is present on the thermochromic layer,
determine one or more locations of the thermochromic layer, and
transmit a message indicating that one or more areas of concern have been detected and including information pertaining to the one or more locations.

5. The wheelchair apparatus of claim 1, wherein the seat comprises a seat body, the thermochromic layer being disposed on or within the seat body.

6. The wheelchair apparatus of claim 1, wherein the seat further comprises a protective layer disposed on the thermochromic layer.

7. The wheelchair apparatus of claim 1, further comprising at least one of a back and an armrest, the at least one of the back and the armrest comprising a second thermochromic layer.

8. The wheelchair apparatus of claim 1, wherein the thermochromic layer comprises at least one of liquid crystals and leuco dyes that produce the first color and the second color.

9. The wheelchair apparatus of claim 1, wherein the surface of the seat further comprises a plurality of temperature sensors arranged in a grid configuration such that each one of the plurality of temperature sensors senses a particular portion of the surface of the seat.

10. A person support apparatus, comprising:
a body; and
a thermochromic layer disposed on a portion of the body that contacts a subject when the subject is supported by the person support apparatus, the thermochromic layer comprising one or more materials that cause the thermochromic layer to:
change to a first color upon application of thermal energy radiating from the subject that corresponds to a baseline body temperature, and
change to a second color that is different from the first color upon application of thermal energy radiating from the subject that corresponds to a temperature that exceeds a threshold difference from the baseline body temperature, the second color indicative of a potential pressure wound.

11. The person support apparatus of claim 10, further comprising one or more imaging devices arranged to capture one or more images of the thermochromic layer.

12. The person support apparatus of claim 11, further comprising:
a processor communicatively coupled to the one or more imaging devices; and
a non-transitory, processor-readable memory module containing programming instructions thereon that, when executed, cause the processor to:
direct the one or more imaging devices to capture a baseline image of the thermochromic layer,
store the baseline image,
receive an indication that the subject is no longer supported by the person support apparatus,
direct the one or more imaging devices to capture an additional image of the thermochromic layer, and
compare the baseline image with the additional image to determine whether any elevated temperature indicators are present.

13. The person support apparatus of claim 12, wherein the programming instructions that, when executed, cause the processor to determine whether any elevated temperature indicators are present further cause the processor to:
determine the first color of the thermochromic layer,
determine that the second color is present on the thermochromic layer,
determine one or more locations of the thermochromic layer, and
transmit a message indicating that one or more areas of concern have been detected and including information pertaining to the one or more locations.

14. The person support apparatus of claim 10, further comprising a protective layer disposed on the thermochromic layer.

15. The person support apparatus of claim 10, wherein the thermochromic layer comprises at least one of liquid crystals and leuco dyes that produce the first color and the second color.

16. The person support apparatus of claim 10, further comprising a plurality of temperature sensors disposed on the body and arranged in a grid configuration such that each one of the plurality of temperature sensors senses a particular portion of the body.

17. A wheelchair apparatus, comprising:
a seat comprising a surface that contacts a subject when the subject is sitting in the wheelchair apparatus;
a plurality of temperature sensors disposed on the surface of the seat, the plurality of temperature sensors arranged in a grid configuration such that each one of the plurality of temperature sensors senses a particular portion of the surface of the seat;
a wheelchair control system comprising a processor and a non-transitory, processor-readable memory module containing programming instructions thereon that, when executed, cause the processor to:
receive baseline temperature data from at least one of the plurality of temperature sensors, the baseline temperature data indicating a first temperature sensed when the subject sits in the wheelchair apparatus;
receive subsequent temperature data from at least one of the plurality of temperature sensors;
determine if the subsequent temperature data indicates a temperature that exceeds a threshold difference from the baseline temperature data; and
when the subsequent temperature data indicates the temperature exceeding the threshold difference, determine a location of the at least one of the plurality of temperature sensors and transmit a notification pertaining to the exceeded threshold and the location.

18. The wheelchair apparatus of claim 17, further comprising a display communicatively coupled to the processor, wherein the display provides the notification pertaining to the exceeded threshold and the location.

19. The wheelchair apparatus of claim 17, further comprising a protective layer disposed over the plurality of temperature sensors.

20. The wheelchair apparatus of claim 17, wherein each of the plurality of temperature sensors is a non-contact sensor or a contact sensor.
